(19) United States
(12) Patent Application Publication
(10) Pub. No.: US 2005/0143792 A1
(43) Pub. Date: Jun. 30, 2005

(54) Hair treatment method

(76) Inventor: Harvey Jay, Scarsdale, NY (US)

Correspondence Address: R. Neil Sudol, 714 Colorado Avenue, Bridgeport, CT, 06605-1601, US

(21) Appl. No.: 10/746,418

(22) Filed: Dec. 24, 2003

(51) Int. Cl. A61N 5/06

(52) U.S. Cl. 607/88

(57) ABSTRACT

A hand held device generates a predetermined number of pulses of light having a predetermined electromagnetic spectrum, a predetermined duration, a predetermined inter-pulse interval, and a predetermined total energy. The pulse sequence is delivered to a skin surface to stimulate hair growth by energizing the hair follicles. The light may be absorbed by endogenous chromophores of the dermal tissues and/or by exogenous chromophores delivered to the dermal tissues. The device is used periodically to apply the pulses of light to the selected skin surface at total energy levels per session that are much lower than the energies used in hair removal treatments.

### BACKGROUND OF THE INVENTION

This invention relates generally to a hair treatment process. More particularly, this invention relates to a method for stimulating or enhancing hair growth.

The use of light to remove unwanted hair is well known. As discussed in U.S. Pat. No. 6,280,438, hair may be removed from selected skin surfaces by the application of intense, wide area, pulsed electromagnetic (light) energy. According to the methodology of U.S. Pat. No. 6,280,438, the energy heats the hair and coagulates the tissue around the hair and follicle without damaging the healthy skin. Pursuant to that prior art disclosure, it is preferable to provide an optically transparent water based gel on the skin prior to treatment with the electromagnetic energy. The gel cools the epidermis but is applied so as not to enter the cavity around the hair follicle, and thus does not cool the hair and the hair follicle. The applied energy then coagulates the hair without damaging the skin.

U.S. Pat. No. 6,280,438 teaches the use of incoherent polychromatic radiation in a wavelength range that penetrates into the skin without being highly attenuated. It is indicated in the patent that wavelengths shorter than 550 nm are not useful because they will be highly attenuated before reaching the lower parts of the hair follicles. Instead, wavelengths in the range of 550 to 630 nm are heavily absorbed by blood and can therefore be used to coagulate the vessels that feed the hairs. Additionally, longer wavelengths, in the range of 600 to 1100 nm have a very good penetration into non-pigmented skin and can be used to couple to the melanin of the hair.

U.S. Pat. No. 5,885,273 discloses a method of removing hair that includes producing a plurality of pulses of incoherent electromagnetic energy, which is filtered in accordance with the color of the hair being removed. A flashlamp produces pulses having delays on the order of 0.1 msec to 100 msec, and an energy fluence on the order of 10 to 100 J/cm². Energy that has a wavelength of less than 500 nm or 600 nm and greater than 1300 nm is preferably filtered out. Light is applied to the treated area in either a long pulse or in a sequence of pulses separated by a delay. The delay and/or pulse length is preferably controlled by the operator to provide enough heat to remove the hair but not enough heat to damage the skin. For example, the pulse length or delay between the pulses should be more than the cooling time of the gel-covered epidermis and less than the cooling time of the hair and follicle. Specifically, a pulse length of 50 msec if a single pulse is used or a delay of 50 msec between the pulses if a pulse sequence is used are appropriate values.

In brief, the art using electromagnetic radiation such as pulses of incoherent light is intended to permanently remove hair from selected skin surfaces. The light pulses have parameters such as spectral dispersion, pulse duration and total energy that are selected to destroy the hair follicles in the selected skin area. Understandably, such methods carry a certain amount of risk that the skin may be damaged. Accordingly, the prior art methods of hair depilation are typically intended for use by trained cosmetologists and other professionals.

It has been disclosed that laser light is useful in the stimulation of hair growth. U.S. Pat. No. 6,497,719 in particular discloses a hand-held laser device that stimulates hair growth. The device provides distributed laser light to the scalp while simultaneously parting the user's hair to ensure that the laser light contacts the user's scalp.

### OBJECTS OF THE INVENTION

An object of the present invention is to provide a method for treating hair to stimulate or enhance hair growth.

It is a related object of the present invention to provide such a method that is safe for home use, so that individuals may use the method on their own scalp.

It is a related object of the invention to provide a method for hair growth treatment, which may replace current home-based methods, for instance, of using drugs or chemicals.

These and other objects of the present invention will be apparent from the drawings and descriptions herein. It is to be understood that each object of the invention is achieved by at least one embodiment of the invention. It is not necessarily the case that any embodiment achieves all of the objects of the invention.

### SUMMARY OF THE INVENTION

A hair treatment method comprises, in accordance with the present invention, generating a predetermined number of pulses of light each having a predetermined electromagnetic spectrum extending over a range of wavelengths, and directing the pulses of light towards a skin surface on which it is desired to grow hair. The pulses have at least one pulse duration and a total energy all predetermined to stimulate growth of hair along the skin surface.

The pulses of light optionally include at least one wavelength absorbable by a chromophore in a person's dermal tissues. The absorption of the light energy is believed to result in microscopically localized heating, which in turn stimulates cellular metabolic activities, leading to hair growth. The application of light energy to a skin surface as described herein is believed to induce localized heating via mechanisms or routes other than photon absorption, for example, by scattering and other kinds of interaction between light energy and matter.

It is contemplated that the light treatment is repeated periodically, for instance, daily or weekly. In some cases, the periodic treatment continues as long as the individual wishes to enjoy stimulated hair growth. In other cases, periodic treatment is required over a limited period only, for instance, several months to a year. In such cases, it is believed that changes induced in the activity of the hair follicles are sufficient to be self-perpetuating.

Pursuant to the present invention, the light is applied via a hand-held instrument, preferably in multiple passes over any particular skin surface where stimulated hair growth is desired. The multiple passes facilitate the application of light to all parts of the target skin surface, for instance, where there is already some hair on the skin surface. The multiple passes may be effectuated with motion in different directions to facilitate access to different parts of the scalp. The selected light treatment parameters may be the same for each pass or may vary from pass to pass. In addition, the passes may follow immediately after one another or may be spaced by an interval during which, for instance, the light treatment device is used to apply light pulses to another area of the user's skin. An advantage of multiple passes is that the total power applied to a given skin surface may be reduced relative to that needed for accomplishing the desired hair growth stimulation by a single pass or light treatment. For example, instead of a single pass of 20 Joules/cm², two passes of 10 Joules/cm2 apiece could effectively stimulate hair growth.

In one embodiment of the present invention, hair growth is stimulated by the application of light without the application of exogenous chromophores for light absorption. As discussed above, the contemplated results may be attained via light absorption by endogenous chromophores particularly including hemoglobin or by other light-matter interaction such as scattering.

The pulse parameters, namely, the pulse number, the pulse duration(s), the inter-pulse interval(s), the total energy and the spectral distribution(s), are selected in concert to stimulate the growth of hair on the treated skin surface. It is hypothesized that the present method works to stimulate hair growth by providing increased energy and increased blood flow (increased oxygen and nutrients) to the follicles.

The light used in a hair treatment method in accordance with the present invention is preferably incoherent and produced by a flashlamp or other source of incoherent electromagnetic radiation. Alternatively, the light may be coherent and produced by a laser source. In the former case, the electromagnetic spectrum of the light pulses is a band of wavelengths, while in the latter case, the electromagnetic spectrum of a light pulse delivered at one time is a single wavelength. In the former case filters may be used to limit the band of transmitted wavelengths, while in the latter case the laser source may be adjustable or tunable for producing wavelengths of different frequencies. In any event, the light energy applied may include one or more wavelengths absorbable by an endogenous chromophore, such as hemoglobin, in the dermal tissues surrounding the hair follicles.

Pursuant to a feature of the present invention, an exogenous chromophore may be added to the person's dermal tissues, for example, through the epidermis via a cream or gel or direct injection, via the circulatory system (ingestion or injection), or via absorption through the airways. Where the exogenous chromophore is deposited dermally through the epidermis, the chromophore medium (cream or gel) may be massaged into the skin surface or, alternatively or additionally, injected directly into the dermis. The same carbon-based chromophores used in conventional hair removal procedures may be used in the present hair growth stimulation methodology. However, the energy levels in the present method are significantly lower than the energy levels used in hair removal. The rate of energy application for hair growth stimulation pursuant to the present invention is preferably about 1% to about 95% and more preferably about 10% to about 80% of the lowest power level used in hair removal methods.

In accordance with the present invention, a user determines a beneficial or optimal group of light application parameters using trial and error. A hand-held light application device may be programmed to have a discrete number of possible parameter combinations. The user selects from among these parameter combinations. Alternatively, a light application device may provide substantial ranges of user adjustability for each of several parameters including pulse duration, light intensity, inter-pulse interval, number of pulses, and total applied energy per pass. In this case, the device may be provided with a failsafe override mechanism for ensuring that the rate of light application does not exceed levels that would damage hair or skin tissues.

Pursuant to a feature of the present invention, the total light that is applied to a target skin surface during any single treatment session is preferably less than approximately 20 Joules/cm² and more preferably less than 10 Joules/cm². Where multiple passes are contemplated, each pass applies a proportionately reduced total energy. Thus, where three passes are contemplated, each pass might provide only 3-5 Joules/cm² to the target skin surface. Where an exogenous chromophore is utilized, the total energy applied is also reduced. For instance, in the case of a single pass and the utilization of an exogenous chromophore, the total energy applied might again be 3-5 Joules/cm². Where an exogenous chromophore such as melanin or carbon black is applied, the heating of the follicles and the stimulation of hair growth may result from light absorption by both hemoglobin and the exogenous chromophore.

A method pursuant to the present invention may entail the use of a template or mask on a person's scalp to guide the user in the application of light. During a calibration period, the user may test different parameter combinations via respective apertures in the template or mask. The template or mask is used later to compare the different areas to gauge effective of the different tested parameter combinations. It is to be noted, however, that an individual's hair may grow at different rates at different locations on his or her scalp. Thus, the testing of different parameter combinations should be effectuated within a localized area on the scalp.

Where a template or mask is used, the template may be provided with markers for enabling the user to place the template on his or her skin at the same location for each treatment and each treatment effectiveness review. The markers may permit the user, for example, to locate the template with respect to physiognomic features of the user's head. Alternatively or additionally, the template or mask may be provided with physical structure for temporarily mounting the template or mask in a particular position on a person's head. The template or mask may take the form of a skullcap with cutouts. The cutouts may be established during the manufacturing process or formed by the user.

The present invention contemplates a periodic application of low-energy light to a skin surface. A user may begin with light applications every day or every other day. As hair growth commences, the interval between successive applications may increase to several days or a week.

It is advisable, at least initially as the hair starts growing, to cut the hair fairly short to facilitate the impingement of the light energy on the skin surface.

The present invention generally contemplates the application of low-energy incoherent light to the scalp region. However, the method may be used in other areas of the skin, if hair growth be desired in such areas, including leg, underarm, chest areas, etc.

The method of the present invention may be carried out using hand held hair-removal devices essentially of prior art designs, for instance, with a light source such as a flashlamp, a reflector, one or more lenses, and an application interface such as a skin-contacting crystal. These prior art designs need be modified only to permit the application of light at energies lower than those used to remove hair. A device dedicated to stimulating hair growth may be constructed to so limit the energy applied that the device that hair removal could not be effectively achieved.

The crystal may function as a cooling element. Alternatively, a separate cooling medium such as a gel may be applied to the skin surface prior to the light application. However, it is preferred that the energies applied are of such low density that special measures for protecting the skin surface are unnecessary.

Accordingly, the present invention contemplates the use of a hand held device for generating a predetermined number of pulses of light having a predetermined electromagnetic spectrum possibly including at least one wavelength absorbable by an endogenous chromophore (e.g., hemoglobin) in a person's dermal tissues and for applying the pulses of light to a skin surface to stimulate follicle activity, the pulses having one or more predetermined durations, one or more predetermined inter-pulse intervals (if number of pulses is greater than one), and a predetermined total energy.

The inter-pulse interval (where the number of pulses is greater than one) may, in different applications of the invention, be anywhere from 1 millisecond to 2 seconds. Generally, the smaller the inter-pulse interval, the greater the risk of damage to the skin. Thus, the smaller inter-pulse intervals should be used only in professional settings. In home-based embodiments of the invention, the inter-pulse interval of a light treatment is preferably greater than 300 msec. An inter-pulse interval of such a magnitude works to prevent inadvertent damage to the hair and the follicles, as well as to the epidermis. Preferably, the inter-pulse interval is between 300 msec and about 1 sec. An inter-pulse interval of 400-500 msec is effective.

The total energy applied may be anywhere from 0.001 Joule per square centimeter of treated skin surface to about 20 J/cm². Preferably, the total energy applied is between approximately 0.01 J/cm² and approximately 18 J/cm² of the skin surface. More preferably, the total energy applied is between approximately 0.1 J/cm² and approximately 10 J/cm² of the skin surface. Generally, the higher energies are more appropriate for persons of light skin color than dark skin color. Concomitantly, the lower energies are more appropriate for persons of dark skin color and not light skin color.

Generally, it is contemplated that devices used in a method pursuant to the present invention will require a selection of a maximum or total energy to be applied to a skin surface. This requirement typically entails some restriction on the user's freedom in selecting the magnitudes of other pulse parameters. In a simple device, the user may be able to select only one pulse parameter, namely the total energy. Such a device might, for instance, have high, medium and low settings. In a more complex device, setting of the total energy applied by a pulse sequence will limit the range of options available to the user in setting the other parameters. For instance, once the user selects the total energy and the pulse duration, the number of pulses is determined, provided that the rate of energy production or intensity is not adjustable. If the intensity is adjustable, the user will have some leeway in selecting both the pulse duration and the number of pulses. In that case, the light-generating device may automatically control the intensity so that the total energy does not exceed the set value.

The duration of the light bursts or pulses may be as little as 1 millisecond or as great as two seconds. In hair removal procedures utilizing the same kind of light application devices, the shortest durations and the higher energies are recommended for professionally supervised light treatments only. However, in the case of hair growth stimulation, the light pulse parameters such as the pulse durations and total applied energies are controlled to result in low rates of energy application, thereby reducing the risk of damage to the hair, the follicles, and the dermal tissues.

Preferred pulse durations in light applications for hair growth stimulation are relatively long, preferably above approximately 7 msec and more preferably above 100 msec.

Pursuant to one embodiment of the present invention, the light of the pulses is incoherent and the spectrum includes wavelengths between about 300 nm and 1200 nm. Longer wavelengths absorbable by endogenous chromophores such as melanin are used for the stimulation of deeper follicles in darker skin. In some embodiments of the invention, the spectrum of the pulses may be limited to wavelengths between about 300 nm and 550 nm. These embodiments will require a more frequent application of the light energy to stimulate hair growth.

The number of pulses in each pulse sequence or treatment session (as applied to a given skin area) may be between one and ten, while the total duration of a pulse sequence may range between 1 millisecond and 38 seconds.

As indicated above, the present invention contemplates that some adjustment may be made by the user in the particular operational parameters of the light application device. For instance, a simple hand-held device may have a plurality of settings, for instance, high, medium, and low, where one or more of the operational parameters have different pre-established values depending on the setting. Thus, high, medium, and low settings may vary in the number of applied pulses, the pulse duration, the inter-pulse interval, and/or the total energy applied. A user could start with a low setting to see whether hair grows and if not, try the next higher setting. Usually, it is preferable to use the lowest setting which accomplishes the desired result.

It is to be noted that consumer devices may be preprogrammed with automatically operating safety controls which inhibit the user from inadvertently exposing himself or herself to dangerous quantities of light energy. Thus, in a relatively complex consumer product, the user's setting of one parameter at a potentially dangerous value will cause the device either to limit the selectable ranges of one or more other pulse parameters or to automatically adjust pulse parameters to prevent an excessive rate of energy delivery. For instance, the selection of a small inter-pulse interval may prevent the user from selecting a short pulse duration and/or a small number of pulses or, alternatively, may result in an automatic diminution of the intensity (e.g., via engagement of an intensity-reducing filter).

A device for hair treatment comprises, in accordance with a feature of the present invention, a hand-holdable casing, a light generator mounted to said casing, and an applicator mounted to the casing for applying light from the generator to the skin surface. The applicator includes a flexible member at least partially conformable to the topography of the skin surface. The flexible member may take the form of a fluid-filled pouch or a piece of resilient plastic material. In either event, the applicator is at least partially transparent to the light produced by the generator for application to the skin surface.

The present invention provides a method for the stimulation of hair growth. The method is safe for home use. The energies used are sufficiently low to avoid skin damage. The present invention contemplates the use of a light applicator periodically, say, at intervals of a day to a week or more.

It should be understood that professional cosmetic service providers may use the present methodology in professional settings, in spas or salons.

### BRIEF DESCRIPTION OF THE DRAWINGS

FIG. 1 is a block diagram of a light-pulse generating device for use in a method in accordance with the present invention.

FIG. 2 is a block diagram of another light-pulse generating device for use in a method in accordance with the present invention.

FIG. 3 is a schematic side elevational view of a template or mask for use in the method of the present invention.

FIG. 4 is a schematic top plan view of the template or mask shown in FIG. 3.

### DETAILED DESCRIPTION OF THE PREFERRED EMBODIMENTS

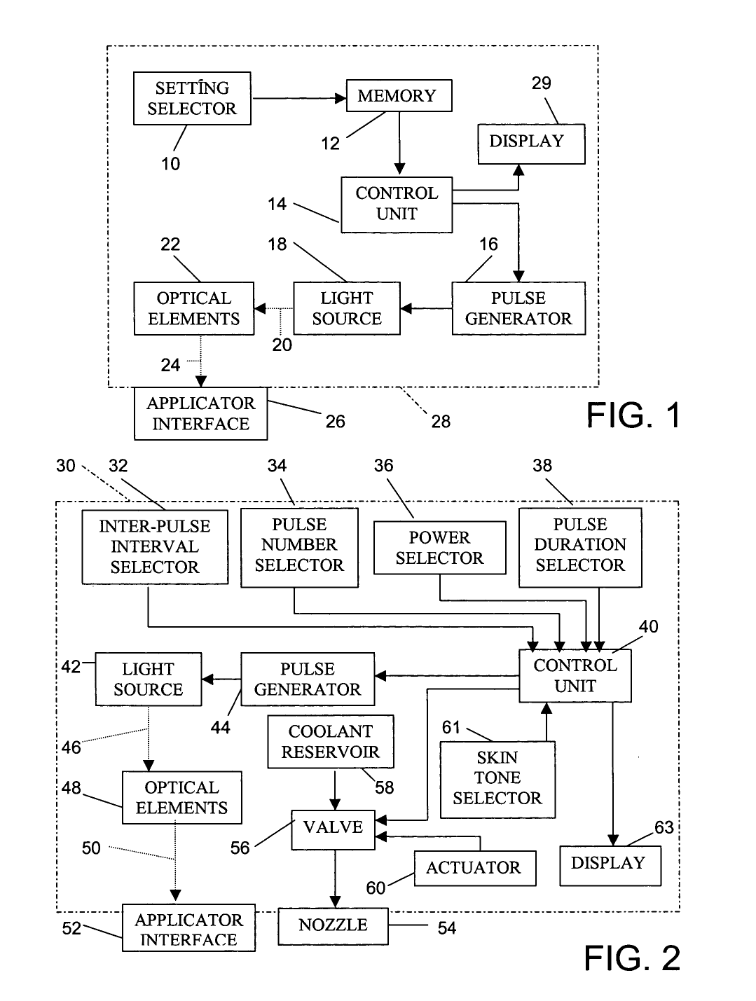

As depicted in FIG. 1, a device for generating light pulses for application to a skin surface in a hair treatment process includes a manually operable setting selector 10 connected at an output to a memory 12 in turn connected at an output to a control unit 14. Memory 12 stores pre-established combinations of light pulse parameters including pulse width or duration, inter-pulse interval or delay time, pulse number, light intensity, and total treatment energy. Control unit 14 may be a microprocessor or a special logic circuit connected to a pulse generator 16 for inducing the generator to produce a sequence of electrical control pulses fed to a source 18 of incoherent light energy. Source 18 produces light with a spectral distribution including wavelengths between 500 nm and 1200 nm. Control unit 14 may be connected directly to source 18 where the source incorporates means for varying pulse parameters pursuant to encoded instructions. Light source 18 (as well as the entire light pulse applicator) may take any known form such as those disclosed in U.S. Pat. No. 6,280,438 and U.S. Pat. No. 5,885,273. Thus, light source 18 may be a Xenon flashlamp.

Light 20 generated by source 18 is directed through an array of optical elements 22 that may include one or more reflectors, lenses, and filters (not separately shown). Where an adjustable filter is included, control unit 14 may be connected to the filter for operatively modifying the action thereof. For instance, in the case of an adjustable neutral density filter, control unit 14 may induce a change in the filter density to control the intensity, and therefore the power, of the light applied to a selected skin surface.

In the case of multiple wavelengths of light being produced, an adjustable filter may be included in the optical elements 22 and/or the applicator interface 26. These filters can block undesired wavelengths and allow desired wavelengths to pass. Low end filters that block lower or shorter wavelengths, high end filters that block higher or longer wavelengths or band pass filters that block some high or some low end wavelengths may be utilized.

Light 24 leaving the optical array 22 is delivered or applied to a skin surface via an applicator or interface element 26 exemplarily taking the form of a crystal. U.S. Pat. No. 6,280,438 and U.S. Pat. No. 5,885,273 disclose kinds of applicators or interfaces utilizable in the device of FIG. 1 (or 2). Applicator or interface element 26 may function in part to cool the skin surface prior to, during, and/or after a light application procedure. Cooling may be accomplished by using a crystal-type applicator or interface 26, with or without a layer of gel, as described in U.S. Pat. No. 6,280,438 and U.S. Pat. No. 5,885,273. Alternatively or additionally, cooling may be accomplished by spraying a coolant on the skin surface or by blowing air or other gas on the skin surface. In the former case, the light application device is provided with a reservoir of coolant fluid, an ejection mechanism or pump and a nozzle. In the latter case, the device is provided with a pump or compressor and a nozzle for directing a jet of air at the skin surface being treated. The elements of FIG. 1 are encased in or mounted to a housing or casing 28 of a size and configuration enabling the pulse generation device to be hand held and easily manipulated for purposes of optically treating different skin surfaces of the individual user.

The device of FIG. 1 is preprogrammed to produce light pulses in any of several settings, each setting being defined by a respective combination of particular operational parameters including pulse duration, inter-pulse interval, pulse number, and intensity or total energy. For instance, the device may have a plurality of settings, for instance, high, medium, and low, which vary in the number of applied pulses (e.g., 3, 2, 1), the pulse duration (100 msec, 60 msec, 10 msec), the inter-pulse interval (300 msec, 400 msec, 500 msec), and/or the total energy applied (10 J/cm², 5 J/cm², 1 J/cm²). A user could start with a low setting to see whether the hair grows and if not, try the next higher setting. Usually, it is preferable to use the lowest setting which accomplishes the desired result. Generally, dark skin is more effective at light absorption that light skin and should be treated with lower settings (e.g., less overall energy) than light skin.

Setting selector 10 has a plurality of discrete settings differentiated at least on the basis of skin tone. Different skin settings may include, for instance, dark skin, medium-tone skin, and light skin, or black, coffee, brown, tan, cream, and white.

Control unit 14 may be connected to an LCD or other readable display 29 for communicating to the user a recommended treatment schedule or program in response to the entries made by the user via selector 10. Depending on the user input, the read-out on display 29 may indicate such schedules as twice daily, once a day, once every other day, weekly, etc. The operational parameters, including pulse duration, inter-pulse interval, pulse number, and intensity or total energy, are selected by control unit 14 to conform to the recommended treatment schedule.

A more advanced or complex device is illustrated in FIG. 2. This device includes a housing or casing 30 having manually actuatable input elements 32, 34, 36, and 38, such as rotary knobs or a solid-state touch screen, which enable a user to individually select multiple operating parameters. Input elements or selectors 32, 34, 36, and 38 are an inter-pulse interval selector, a pulse number selector, a power or energy selector, and a pulse duration selection, respectively. Another selector (not shown) could be for intensity adjustment, while a further selector may be provided for adjusting a light source 42 or a filter in optical elements 48 and/or an applicator 52 for modifying the wavelength band delivered to the target skin surface. Selectors 32, 34, 36, and 38 are operatively tied to a control unit 40 such as a microprocessor or hard-wired log circuit. Control unit 40 regulates the operation of light source 42 such as a conventional flashlamp, either directly or indirectly via a pulse generator 44. Light 46 from source 42 is transmitted along a path through optical elements 48 optionally including one or more reflectors, lenses, and filters (not separately shown). Light 50 at an output of the optical array 48 is applied to a skin surface via applicator or interface element 52. Applicator or interface element 52 may take the form of a crystal block, a flexible plastic element, and/or a transparent or translucent pouch filled with a transparent or translucent fluid such as a gel or a liquid. In the case of the flexible applicator element or the fluid-filled pouch, applicator or interface element 52 conforms at least partially to the changing topography of the skin surface under treatment, thereby facilitating the retention of gel (when utilized) between the applicator or interface 52 and the skin surface. This result decreases the likelihood of overexposed or burned skin and generally provides a more uniform application of light with a uniformity of cooling. Safety is enhanced, while the outcomes to successive procedures become increasingly standardized.

Where the applicator element or interface 52 (or 26) is a fluid-filled pouch, the fluid may comprise a suspension or solution of hydrogel particles. More particularly, the pouch may further comprise a polymeric envelope or casing that contains the hydrogel particles and is provided with one or more flaps, flanges, brackets, or supports for facilitating mounting of the applicator element or interface 52 to housing or casing 30. The flaps, flanges, brackets, or supports may be removably attached to housing or casing 30 for facilitating the attachment of replacement interface cartridges to the housing or casing. As discussed below, the hydrogel-containing fluid may be exuded onto a skin surface through perforations in a polymeric envelope or casing of applicator element or interface 52 (or 26).

Where applicator element or interface 52 (or 26) is made in substantial part as a mass of a flexible material, that material may take the form of a transparent or translucent hydrogel such as poly(ethylene oxide), poly(vinyl alcohol), polyvinylpyrrolidone, and poly(hydroxyethyl methacrylate). The polymer networks of the hydrogel material are suitably cross-linked to provide a firm cohesiveness to the applicator element or interface 52 (or 26). Other suitable hydrogels are silicone hydrogels used in the contact lens industry, such as (A) fluoroether macromer co-polymerized with the monomer trimethylsiloxy silane and N,N-dimethyl acrylamide in the presence of a diluent and (B) the monomer polydimethylsiloxane co-polymerized with the hydrophilic hydrogel monomer N-vinyl pyrolidone. A hydrogel applicator element or interface 52 (or 26) may be annealed to a solid and substantially rigid support element for removably securing the hydrogel applicator element or interface to housing or casing 30 (or 28).

Housing or casing 30 may be provided with a fluid-filled chamber (not shown) in heat- and light-transmitting contact with light source 42. Optical elements 48 may be disposed in this fluid. The fluid may be that of the applicator element or interface 52 or may be a separate aliquot of fluid disposed in a separate chamber.

Applicator element or interface 52 may be placed in a refrigerated chamber prior to use in a hair treatment method as disclosed herein. This procedure is especially convenient when the applicator element or interface 52 is removable from housing or casing 30. However, if removal is not feasible, the entire device of FIG. 1 or 2 may be placed in the cooling space.

As an alternative to the flexible applicator or fluid-filled pouch, applicator or interface element 52 may include a plurality of independently movable substantially rigid transparent or translucent members (not shown) that collectively define a tissue-engaging surface. These independently movable members may take the form of closely packed pins or plates that are each independently spring biased to an extended position. Pressure of topographical dermal features against the independently movable pins or plates during use of the light-pulse generating device causes the pins or plates to move in opposition to the respective spring bias, to thereby conform the tissue engaging surface of the light-pulse generating device to the skin surface under treatment. The independently movable pins or plates may be disposed in a holder or bracket attached to the housing or casing 30 and retained there by friction forces.

Where applicator 52 (or 26) includes a fluid- or gel-filled pouch, the pouch (52) may be provided with perforations on a skin-contacting surface for exuding the fluid or gel for cooling purposes. Alternatively, as shown in FIG. 2, the light pulse device may be provided with a fluid dispenser such as a spray nozzle 54 connected to a valve 56 downstream of a pressurized coolant reservoir 58. In response to an operation of a manual actuator 60 or in response to signals from control unit 40, valve 56 enables a flow of coolant from reservoir 58 to nozzle 54 for application to a selected skin surface. In the event that applicator or interface element 52 is a bag or pouch, reservoir 58 and valve 56 may be connected to the applicator or interface element for supplying a gel or fluid coolant thereto.

In one embodiment of the device of FIG. 2, suitable for professional but not home use, inter-pulse interval selector 32 provides for intervals in a range from 1 msec and 2 seconds, whereas pulse number selector 34 is enabled for pulse sequences of one to ten pulses. In addition, power selector 36 permits treatment energies between 0.1 Joule per square centimeter of skin surface and 200 Joules per square centimeter, while pulse duration selector 38 enables pulses of 1 msec to 2 seconds in length. Total pulse sequence duration, from the beginning of the first pulse to the termination of the final pulse, ranges from 1 msec to 38 seconds. The various pulse sequence parameters may be selectable from sets of discrete values or, alternatively, from continuous ranges.

In the device of FIG. 2, the various parameters are typically not completely independent inasmuch as the total energy selected will function as a constraint on the ranges available for the other parameters, that is, the total energy selected will serve to regulate or circumscribe the ranges available to the user for the other pulse sequence parameters. Where the device of FIG. 2 has no intensity adjustment capability, a selection of the total energy and the pulse duration may determine the number of pulses. Similarly, a selection of the total energy and the number of pulses may determine the pulse duration. If the intensity is an adjustable parameter, once the total energy has been chosen, the user will be able to select the magnitudes of two of the three parameters, pulse duration, intensity and number of pulses. The inter-pulse interval is related to the rate at which radiant energy is applied to a skin surface and may accordingly be subjected to some programmed control. Longer pulse durations and/or delays will deliver energy at a slower rate (total energy is distributed over longer time) and therefore be safer to use with higher energy levels. Preferably, the total energy is always a selectable parameter and is best selected prior to the setting of the other parameters. However, the device of FIG. 2 may be preprogrammed to limit the rate at which radiant energy is applied to a skin surface, which will force restrictions on the user's ability to select pulse parameter values.

In an alternative embodiment of the device of FIG. 2, suitable for home use, inter-pulse interval selector 32 enables a selection of intervals ranging from 200 msec to 2 seconds, while power selector 36 enables treatment energies between 0.1 J/cm² and 20 J/cm². Preferably, the pulse duration and the number of pulses available for selection are restricted so as to prevent the user from delivering energy at too high a rate. If the user selects a large pulse number, the pulse duration is necessarily short, whereas a small number of pulses forces a longer pulse duration in order to achieve the selected total energy. It is preferable to use a larger number of pulses and a smaller pulse duration in order to limit the rate at which light energy is applied to a skin surface. Pulse number selector 34 may therefore enable a selection of three to ten pulses per pulse sequence, while pulse duration selector 38 enables a selection of pulses lasting 1 msec to 500 msec. The various pulse sequence parameters may be selectable from sets of discrete values or, alternatively, from continuous ranges.

As further illustrated in FIG. 2, the light pulse applicator of that drawing figure may include an additional selector 61 for enabling the user to load a skin tone value into control unit 40. Setting selector 61 may take the form of a knob, a keypad, or manual switch enabling the user to enter a skin tone value from a plurality of possible values. The possible selections may be qualitative descriptions: black, coffee, brown, tan, cream, white, etc. Alternatively, the possible skin tone selections may be quantitative where the integer 1 corresponds to the whitest skin tone while the numeral 10 represents the darkest possible skin tone.

Control unit 40 may be connected to an LCD or other readable display 63 for prompting the user as to information to be input via selectors 32, 34, 36, 38 and 61 and for communicating to the user a recommended treatment schedule or program in response to the entries made by the user via selector 61. Control unit 40 may also use display 63 to alert the user as to impermissible parameter combinations. Depending on the skin tone input received via selector 61, control unit 40 may reject certain light parameter selections made by the user. The reason for rejecting the user's selection of light pulse parameters may be communicated to the user via display 63. Ranges of permissible values may be displayed to facilitate the user's selection. The permissible ranges for outstanding parameters will vary depending on the user's prior selections as discussed above.

In response to user input of skin tone, control unit 40 may indicate on display 63 a plurality of possible light treatment schedules. The user may actuate selector 61 to choose a preferred treatment schedule. This choice is preferably made prior to the selection of one or more light pulse parameters via selectors 32, 34, 36, and 38. The available ranges of the remaining parameters will be limited in accordance with the user's selection of treatment schedule.

Generally, light treatment schedules are determined automatically in accordance with a user's input to the light treatment device. To the extent that the user is allowed leeway in selecting individual pulse parameters, the device may impose limitations in accordance with the selected skin tone, as well as one or more pulse parameters initially selected by the user.

A person uses the device of FIG. 1 or 2 to apply pulses of light to a skin surface for purposes of energizing or stimulating hair follicles to produce hair at an enhanced rate. The user first performs a calibration or initialization procedure to determine an effective, if not optimal, pulse setting for the particular skin area where enhanced hair growth is desired.

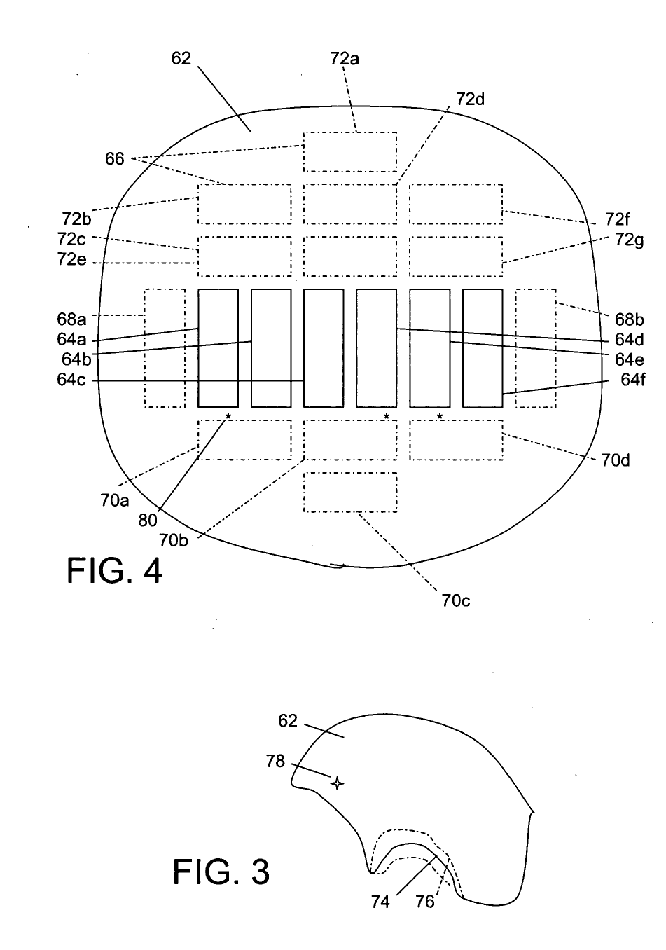

A calibration or initialization procedure may entail the use of a template or mask 62 (FIGS. 3 and 4) exemplarily in the form of a skullcap positioned on a person's scalp to define plural target areas and to guide the user in the application of light of different parameter combinations to the respective areas. The template or mask 62 may be provided with several preformed apertures 64a, 64b, 64c, 64d, 64e, 64f adjacent to one another. Alternatively, apertures 64a, 64b, 64c, 64d, 64e, 64f may be formed by the user by cutting the template or mask 62 or by severing the template material along preformed score lines as at 66. Template or mask 62 may include preformed apertures 64b, 64c, 64d, and 64e together extending over the sagital crest of the user, scored areas 68a and 68b and preformed apertures 64a and 64f located over the temporal lobes of the user, further scored areas 70a through 70d located in a forehead region, and additional scored areas 72a-72g located over a rear (occipital) portion of the head.

In an alternate configuration, different templates or masks may be formed with groups of preformed or pre-scored apertures only in certain locations, for instance, only over the forehead, or only over the sagital crest, or only over a temporal lobe, etc. As indicated above, the user may cut the openings out in accordance with the location or locations where the user wishes to experience enhanced hair growth.

The apertures of any template, e.g., template or mask 62, should be fairly restricted in size. For instance, where the apertures are rectangular or oval, dimensions of about one-half inch wide by one inch long are acceptable for most purposes.

During the calibration period, the user may test different parameter combinations via respective apertures 64a-64f, 68a, 68b, 70a-70d, 72a-72g in the template or mask 62. Thus, for a period of one week to a couple of months, the user applies light with one set of parameters to a skin surface showing through a first aperture, for instance, aperture 64a in the template, and subsequently applies light with another set of parameters to a skin surface showing through either adjacent aperture 62b or, more preferably, the symmetrically located aperture 64f. During the calibration period, once the user places the template 62 over his head, he inspects the respective skin surfaces at apertures 62a and 62f (or 62b) and compares the hair growth. Once sufficient time has passed, the user can determine which of the two combinations of parameters results in the greatest acceleration of hair growth. Because an individual's hair is liable to grow at different rates at different locations on his or her scalp, it is important that the apertures in the template be spaced close to one another.

Where a template or mask 62 is used, the template may be provided with markers for enabling the user to place the template on his or her skin at the same location for each treatment and each treatment effectiveness review. The markers may take the form of arcuate cutouts 74 for locating the template or mask 62 on the user's ears. To that end, the template may be provided with preformed score lines 76 or may otherwise be cut by the user to customize the ear cutouts 74 to the individual user's skull. Alternatively, or additionally, the manufacturer or the user may provide template or mask 62 with markers 78 that permit the user to locate the template with respect to physiognomic features such as the inner edges of the eyebrows or the eyes and the front edges of the ears.

A user may begin with light applications every day or every other day. As hair growth commences, the interval between successive applications may increase to several days or a week. After a selection of optimal parameters has been completed, the user typically, at least initially as the hair starts growing, cuts the hair fairly short to facilitate the impingement of the light energy on the skin surface. If longer hair is desired, a device as disclosed in U.S. Pat. No. 6,497,719 may be used to move the hair during a light application session.

It is to be noted that to treat an area of the scalp to stimulate hair growth may not require the direct application of light to every area of the scalp. Where light pulses as described herein are applied to a preselected target area of the scalp, areas adjacent to that target area may experience hair growth. For instance, if light is applied directly through apertures 64a, 64d, and 64e of template or mask 62, the areas of the scalp at apertures 62b, 64c, and 64f may experience some hair growth enhancement. Of course, the result will depend not only on the individual user but also on the settings (light pulse parameters) selected by the user for the different apertures 64a, 64d, and 64e. During testing and use, the user may mark template 62 as at 80 as a reminder as to which apertures 64a, 64d, 64e are being used for light pulse application.

During the calibration or initialization stage, the user should first select a low-energy pulse sequence to determine whether that sequence is effective in stimulating hair growth in a selected skin region. The individual may find that a given setting does not adequately stimulate the hair. In such cases, the individual should retry the calibration or initialization procedure using a higher-energy setting.

Using the device of FIG. 1, an individual will first select a low setting to determine whether that low setting is effective in hair growth stimulation. If not, a next higher or medium setting may be tried. Generally, higher settings will be used only as the circumstances warrant, for instance, if the hair fibers to be grown are likely thick (as determined from other areas on the user's body) and the skin is light.

In determining optimal settings with the device of FIG. 2, a user should choose initial parameter values which in combination result in the application of small amounts of energy, for instance, in a range around 0.1 j/cm² of the skin surface. Thus, where one or more selected pulse parameters are associated with high treatment energies, other pulse parameters should be selected that are associated with low treatment energies.

Where all the pulse parameters are independently adjustable, lower treatment energies will generally result from settings involving few pulses (say, 1-3 instead of 8-10 pulses), long inter-pulse intervals (more than 300 or even 500 msec), short pulse durations (20 msec or less), low light intensity (if selectable, for example, via an adjustable neutral density filter), and low total energies (less than 10 Joules per square centimeter of skin surface). If a given setting proves to be ineffective, the user might adjust selector 32 or 38 to decrease the inter-pulse interval or increase the pulse length, thereby effectively increasing the power or rate at which the radiant energy is delivered to the target skin surface. Alternatively or additionally, the user might increase the number of pulses via selector 34 or increase the applied energy via selector 36. These adjustments will result in an increase in the rate of applied energy if the total time of the pulse sequence is limited. If the light intensity is separately adjustable, one may increase the power or rate of energy delivery by simply selecting a higher intensity value.

Where the various pulse parameters are not independently selectable, for instance, where the total energy applied is a controlling factor, adjustments made in the parameters for purposes of incrementally enhancing the effectiveness of the device of FIG. 2 will be different from the case of completely independent parameter values. For instance, once the total applied energy and total pulse sequence time have been selected, decreasing the number of pulses will require an increase in pulse length and/or an increase in pulse intensity in order to deliver the same amount of total energy in the fixed time. These changes will increase the effectiveness of the light application inasmuch as the rate of energy delivery is increased. In contrast, once the total applied energy and total pulse sequence time have been selected, increasing the pulse duration will decrease the instantaneous rate at which energy is applied to the target skin surface by decreasing the light intensity.

Since hair growth rates vary from person to person and for different body locations on the same person, each user should note the interval between the first treatment and the appearance of new hair on each skin area. This initial appearance of new hair is in many cases the most reliable indicator of the effectiveness of a given energy level and a given set of light pulse parameters.

Because different skin areas have different grades of hair (different colors, different fiber diameters, different hair densities) and accordingly different kinds of hair follicles, as well as different skin pigmentation, etc., different pulse parameter settings are recommended for different skin areas. For example, different settings may be necessary for the crown of the head and the sides of the head in order to optimize results. In addition, optimal light application schedules may also vary from one skin area to another.

After the user has determined pulse sequence parameters that result in acceptable rates of hair growth for at least one target skin area, the user then treats that target skin surface with pulsed light having the determined parameters, in a treatment schedule with a periodicity or inter-session interval also determined during the calibration or initialization procedure. With the apparatus of FIG. 1, the user starts at a low energy setting and applies the light pulses to one template-defined skin area daily and to another template-defined skin area weekly. If the user determines that new hair appears more rapidly in the skin area treated daily, the user then tests the same low setting again at daily intervals and, say, at four-day intervals and compares the results. The next test involves light application every other day in one skin area and either daily or every fourth day in the other skin area. Some users will that that satisfactory results are obtained with little testing, while others will require testing not only with varying inter-session intervals but also with different pulse parameters, such as the medium and high settings of the apparatus of FIG. 1.

During the initial testing period using the instrument of FIG. 2, the user should starts with a low total applied energy, such as 0.1 J/cm² for dark skin and 0.3 or 0.4 for light skin. Exemplary parameter choices for a low-energy setting include a high pulse number, such as 7-10 pulses per pass, a long inter-pulse interval such as 500 msec or above, and a long pulse duration also 500 msec or greater. Light pulses of this low-energy combination of pulse parameters are applied to a first skin area as defined by a template aperture. A second skin area may be treated with light pulses of the same combination of parameters at a different inter-session interval, or with light pulses having a modified parametric characterization. For instance, the inter-pulse interval and/or pulse duration may be shorter.

This hair growth method contemplates, therefore, the periodic application to a selected skin surface of a pulse sequence having a predetermined number of pulses of light of a predetermined electromagnetic spectrum, a predetermined duration, a predetermined inter-pulse interval, and a predetermined total energy. These pulse sequence parameters are determined in part by the design of the light-generating device used and in part by the selections made by the user. The light treatment stimulates a growth of hair through the selected skin surface at least while light treatment is continued.

The light of the pulses is generally incoherent and the spectrum includes wavelengths between about 300 nm and 1200 nm. However, single wavelengths of laser or coherent light may be delivered at one time, when desired. Higher wavelengths are used for darker skin and for deeper hair follicles.

The light applied to a skin surface by the devices of FIGS. 1 and 2 may include one or more wavelengths absorbable by one or more endogenous chromophores in a person's dermal tissues. One such endogenous chromophore is hemoglobin. However, a form of melanin that is found in the skin tissues may also selectively absorb one or more wavelengths in the applied light spectrum. In a more advanced embodiment the light application device may include a setting or control (not shown) for selecting a spectrum or range of wavelengths appropriate to the user's skin and/or hair color. Absorption of limited amounts of light by the growing hair, particularly at the hair follicles, will serve to stimulate the follicles to produce hair at an increased rate. The devices of FIGS. 1 and 2 are preferably constructed with built-in safeguards to ensure that the applied energy, especially at wavelengths absorbable by chromophores in the hair, is not so strong as to damage or sever the hair fibers. In this regard, it is noted that, for lighter hair, the energy intensity emitted at the one or more natural absorption wavelengths of pheomelanin should be restricted. For darker hair, the energy intensities at the one or more natural absorption wavelengths of eumelanin should be limited. In any event, the devices of FIGS. 1 and 2 may be used without the application of an exogenous chromophore to a target skin surface for light absorption purposes. Hair growth stimulation may be accomplished by light absorption solely by one or more endogenous chromophores. Alternatively, exogenous chromophores may be delivered to the dermal tissues for purposes of enhancing energy absorption and therefore hair growth stimulation.

In other embodiments of a light generation and application device for hair treatment, one or more of the pulse parameters may vary during a single treatment session. For instance, the inter-pulse interval or the pulse duration may increase or decrease from the beginning of a pulse sequence to the end of the pulse sequence. The resulting instantaneous rate of energy application may therefore vary during the pulse sequence.

Listed below are a number of exemplary settings or combinations of operational parameters particularly suitable for home-use and attainable with either the device of FIG. 1 having pre-established settings or parameter combinations or the device of FIG. 2 where the various pulse sequence parameters may be individually adjusted independently of the other parameters. In these examples, the selected numbers of pulses, the selected pulse durations and the selected inter-pulse intervals jointly determine the total times of the pulse sequences. The light-generating device, if necessary to ensure consistency among the listed parameter settings, may automatically adjust the light intensity.

### HOME USE EXAMPLE 1

In a preferred setting or combination of operational parameters suitable for home use, an incoherent light applicator device for stimulation of hair growth generates pulses with a pulse number of three, a pulse duration of 100 msec, an inter-pulse interval of 400 msec, a total pulse energy of 5 J/cm², and a spectral distribution of a commercially available flashlamp, including wavelengths between 500 and 1200 nm.

### HOME USE EXAMPLE 2

A slightly higher setting or combination of operational parameters for an incoherent light applicator device suitable for home use involves a pulse sequence with a pulse number of three, a pulse duration of 100 msec, an inter-pulse interval of 350 msec, a total pulse energy of 5 J/cm², and a spectral distribution of a commercially available flashlamp, including wavelengths between 500 and 1200 nm. Although the total amount of energy is the same as in the first example, the shorter interpulse interval means that the rate of energy transmission to the target skin surface is higher.

### HOME USE EXAMPLE 3

A higher setting or combination of operational parameters for an incoherent light applicator device involves pulses with a pulse number of three, a pulse duration of 75 msec, an inter-pulse interval of 350 msec, a total pulse energy of 10 J/cm², and a spectral distribution of a commercially available flashlamp, including wavelengths between 500 and 1200 nm. In this example, not only is the total energy larger than in the second example, but the rate of energy application is higher owing to the shorter pulse duration.

### HOME USE EXAMPLE 4

An even higher setting or combination of operational parameters for an incoherent light applicator device involves pulses with a pulse number of three, a pulse duration of 75 msec, an inter-pulse interval of 300 msec, a total pulse energy of 11 J/cm², and a spectral distribution of a commercially available flashlamp, including wavelengths between 500 and 1200 nm. The pulse sequence of this example delivers radiant energy at a higher rate than in the third example because of the shorter inter-pulse interval and the slightly higher energy delivered per pulse.

### HOME USE EXAMPLE 5

In a low setting or combination of operational parameters, an incoherent light applicator device produces pulses with a pulse number of four, a pulse duration of 60 msec, an inter-pulse interval of 500 msec, a total pulse energy of 1 J/cm², and a spectral distribution of a commercially available flashlamp, including wavelengths between 500 and 1200 nm. The pulse sequence of this example delivers a small amount of energy, at a low rate (e.g., long inter-pulse interval).

### HOME USE EXAMPLE 6

A slightly higher setting or combination of operational parameters for an incoherent light applicator device involves pulses with a pulse number of four, a pulse duration of 60 msec, an inter-pulse interval of 450 msec, a total pulse energy of 1 J/cm², and a spectral distribution of a commercially available flashlamp, including wavelengths between 500 and 1200 nm.

### HOME USE EXAMPLE 7

A lower setting or combination of operational parameters for an incoherent light applicator device involves pulses with a pulse number of four, a pulse duration of 60 msec, an inter-pulse interval of 550 msec, a total pulse energy of 0.9 J/cm², and a spectral distribution of a commercially available flashlamp, including wavelengths between 500 and 1200 nm.

### HOME USE EXAMPLE 8

Another setting or combination of operational parameters for an incoherent light applicator device involves pulses with a pulse number of two, a pulse duration of 60 msec, an inter-pulse interval of 3500 msec, a total pulse energy of 1 J/cm², and a spectral distribution of a commercially available flashlamp, including wavelengths between 500 and 1200 nm.

### HOME USE EXAMPLE 9

Another setting or combination of operational parameters for an incoherent light applicator device involves pulses with a pulse number of two, a pulse duration of 7 msec, an inter-pulse interval of 300 msec, a total pulse energy of 5 J/cm², and a spectral distribution of a commercially available flashlamp, including wavelengths between 500 and 1200 nm.

The devices of FIGS. 1 and 2 may be provided with a band-pass filter for limiting the spectral distribution of the generated light pulses to wavelengths in a given band, for instance, between 700 nm and 900 nm. Alternatively, a high-pass filter may be used for transmitting to a skin surface only wavelengths greater than a predetermined minimum, such as 550 nm, 750 nm, or 900 nm. The higher the wavelength the more likely the light will penetrate deeply and energize, activate or stimulate cellular and histological elements as deep as the bulb parts of the hair follicles.

Using higher light intensities may also increase depth of penetration. Neutral density or “gray” filters are used sparingly so as to reduce the intensity of the light applied to the selected skin surfaces so as to avoid damage to the hair and follicles that would result in hair removal or growth retardation rather than growth stimulation.

Listed below are a number of exemplary settings or combinations of operational parameters particularly suitable for professional devices. In these examples, the selected numbers of pulses, the selected pulse durations and the selected inter-pulse intervals jointly determine the total times of the pulse sequences. The light-generating device, if necessary to ensure consistency among the listed parameter settings, may automatically adjust the light intensity.

### PROFESSIONAL USE EXAMPLE 1

In a setting or combination of operational parameters suitable for professional use, an incoherent light applicator device for stimulation of hair growth generates pulses with a pulse number of two, a pulse duration of 50 msec, an inter-pulse interval of 350 msec, a total pulse energy of 15 J/cm², and a spectral distribution of a commercially available flashlamp, including wavelengths between 500 and 1200 nm.

### PROFESSIONAL USE EXAMPLE 2

A slightly higher setting or combination of operational parameters for an incoherent light applicator device involves pulses with a pulse number of two, a pulse duration of 50 msec, an inter-pulse interval of 300 msec, a total pulse energy of 15 J/cm², and a spectral distribution of a commercially available flashlamp, including wavelengths between 500 and 1200 nm.

### PROFESSIONAL USE EXAMPLE 3

A lower setting or combination of operational parameters for an incoherent light applicator device involves pulses with a pulse number of two, a pulse duration of 60 msec, an inter-pulse interval of 450 msec, a total pulse energy of 15 J/cm², and a spectral distribution of a commercially available flashlamp, including wavelengths between 500 and 1200 nm.

### PROFESSIONAL USE EXAMPLE 4

A higher setting or combination of operational parameters for an incoherent light applicator device involves pulses with a pulse number of two, a pulse duration of 60 msec, an inter-pulse interval of 450 msec, a total pulse energy of 19 J/cm², and a spectral distribution of a commercially available flashlamp, including wavelengths between 500 and 1200 nm.

### PROFESSIONAL USE EXAMPLE 5

In a relatively low setting or combination of operational parameters for professional use, an incoherent light applicator device produces pulses with a pulse number of two, a pulse duration of 75 msec, an inter-pulse interval of 500 msec, a total pulse energy of 15 J/cm², and a spectral distribution of a commercially available flashlamp, including wavelengths between 500 and 1200 nm.

### PROFESSIONAL USE EXAMPLE 6

A higher setting or combination of operational parameters for an incoherent light applicator device involves pulses with a pulse number of two, a pulse duration of 70 msec, an inter-pulse interval of 450 msec, a total pulse energy of 20 J/cm², and a spectral distribution of a commercially available flashlamp, including wavelengths between 500 and 1200 nm.

### PROFESSIONAL USE EXAMPLE 7

Another higher setting or combination of operational parameters for an incoherent light applicator device involves pulses with a pulse number of two, a pulse duration of 50 msec, an inter-pulse interval of 300 msec, a total pulse energy of 20 J/cm², and a spectral distribution of a commercially available flashlamp, including wavelengths between 500 and 1200 nm.

### PREOFESSIONAL USE EXAMPLE 8

Another setting or combination of operational parameters for an incoherent light applicator device involves pulses with a pulse number of two, a pulse duration of 7 msec, an inter-pulse interval of 300 msec, a total pulse energy of 12 J/cm², and a spectral distribution of a commercially available flashlamp, including wavelengths between 500 and 1200 nm.

An incoherent light applicator device for professional use may also be provided with a band-pass filter for limiting the spectral distribution of the generated light pulses to wavelengths in a given band, for instance, between 700 nm and 900 nm. Again, a high-pass filter may be used for transmitting to a skin surface only wavelengths greater than a predetermined maximum, such as 550 nm, 750 nm, or 900 nm.

The hair treatment method described above with reference to FIGS. 1 and 2 results in at least a temporary stimulation of hair growth rates along an optically treated skin surface. By counting the days to hair reappearance after the protruding hair has been shaved, it is possible to determine a changes in hair growth rate owing to the application of different combinations of light-pulse parameters. A user who starts using the light application process at one inter-application or inter-session interval may subsequently use a longer inter-application interval and still maintain a satisfactory hair-growth rate along the treated skin surface. Of course, the degree of hair growth rate enhancement will vary from person to person and even from skin location to skin location on the same person. For example, two users initially required to apply the pulsed light energy at intervals of one day in order to ensure the appearance of hair on the treated hair surface may find that after several months one user need reapply light energy only every week and the other user need reapply light energy only every other day.

It is to be noted that the hair treatment method described herein contemplates multiple passes over any particular skin surface. The selected light treatment parameters may be the same for each pass or may vary from pass to pass. In addition, the passes may follow immediately after one another or may be spaced by an interval during which, for instance, the light treatment device is used to apply light pulses to another area of the user's skin. An advantage of multiple passes is that the total power applied to a given skin surface may be reduced relative to that needed for accomplishing the desired hair removal by a single pass or light treatment. For example, instead of a single pass of 10 Joules/cm², hair growth could be effectively stimulated by two passes of 5 Joules/cm² apiece.

Although the invention has been described in terms of particular embodiments and applications, one of ordinary skill in the art, in light of this teaching, can generate additional embodiments and modifications without departing from the spirit of or exceeding the scope of the claimed invention. For example, light sources 18 and 42 may take the form of laser sources or light emitting diodes. In that case, if optical elements 22 and 48 include any filters, those filters are neutral density filters for reducing the intensity of the transmitted radiation. Where light sources 18 and 42 are tunable laser sources, then an additional actuator may be provided for frequency selection purposes.

It is to be noted further that the various devices disclosed herein, particularly with reference to the applicator element or interface 26 or 52, may be used for the application of light to a skin surface for purposes other than hair growth stimulation. For example, the devices may be used for temporary or permanent hair removal or skin rejuvenation or blood vessel treatment.

Accordingly, it is to be understood that the drawings and descriptions herein are proffered by a way of example to facilitate comprehension of the invention and should not be construed to limit the scope thereof.

1. A hair treatment method comprising:
generating a predetermined number of pulses of light each having a predetermined electromagnetic spectrum extending over a range of wavelengths;
directing said pulses of light towards a skin surface on which it is desired to grow hair, said pulses having at least one pulse duration and a total energy all predetermined to stimulate growth of hair along said skin surface; and
at least in part owing to the applying of said pulses to respective dermal tissues of said skin surface, stimulating follicles in said respective dermal tissues to grow hair.

2. The method defined in claim 1 wherein said total energy is less than approximately 20 Joules of energy per square centimeter of said skin surface.

3. The method defined in claim 2 wherein said number of pulses is greater than one, said pulses having an inter-pulse interval of at least 300 msec approximately.

4. The method defined in claim 3 wherein said duration is between approximately 1 msec and approximately 2 seconds.

5. The method defined in claim 4 wherein said spectrum includes wavelengths between about 300 nm and 1200 nm.

6. The method defined in claim 2 wherein said total energy is between approximately 0.01 Joules and approximately 10 Joules of energy per square centimeter of said skin surface.

7. The method defined in claim 1 wherein said range of wavelengths includes at least one wavelength absorbable by a chromophore endogenous to said dermal tissues.

8. The method defined in claim 7 wherein said dermal tissues include dermal blood vessels and wherein said chromophore is hemoglobin.

9. The method defined in claim 1 wherein the light of said pulses is incoherent.

10. The method defined in claim 1, further comprising applying an exogenous chromophore to said skin surface prior to the directing of said pulses towards said skin surface, said pulses of light including at least one wavelength absorbable by said exogenous chromophore.

11. The method defined in claim 1, further comprising disposing a template or mask along said skin surface prior to the directing of said pulses of light towards said skin surface, and maintaining said template or mask disposed along said skin surface during the directing of said pulses of light towards said skin surface.

12. The method defined in claim 1 wherein said number of pulses are generated in a plurality of sets, at respective times, the directing of said pulses of light towards said skin surface including directing of the pulses of said sets towards said skin surface at said respective times, thereby effectuating multiple passes over said skin surface.

13. A hair treatment method comprising:
generating first pulses of light each having a predetermined electromagnetic spectrum extending over a range of wavelengths;
directing said first pulses of light towards a first skin area on which it is desired to grow hair, said first pulses being defined by a first set of parameters including number of pulses, pulse duration and total energy;
generating second pulses of light each having a predetermined electromagnetic spectrum extending over a range of wavelengths;
directing said second pulses of light towards a second skin area on which it is desired to grow hair, said second pulses being defined by a second set of parameters including number of pulses, pulse duration and total energy;
comparing hair growth along said first skin area with hair growth along said second skin area to determine which of said first set of parameters and said second set of parameters is a relatively optimal set of parameters for stimulating hair growth;
subsequently generating third pulses of light each having a predetermined electromagnetic spectrum extending over a range of wavelengths; and
directing said second pulses of light towards said first skin area and said second skin area, said third pulses being defined by said optimal set of parameters.

14. The method defined in claim 13 wherein at least one of said first set of parameters and said second set of parameters includes a number of pulses greater than one, said one of said first set of parameters and said second set of parameters including an inter-pulse interval between 1 msec and 2 seconds.

15. The method defined in claim 13 wherein said inter-pulse interval is greater than approximately 300 msec.

16. The method defined in claim 13 wherein said first set of parameters and said second set of parameters each include a total energy of less than approximately 20 Joules of energy per square centimeter of said skin surface.

17. The method defined in claim 13 wherein said first set of parameters and said second set of parameters each include a total energy between approximately 0.01 Joules and approximately 10 Joules of energy per square centimeter of said skin surface.

18. The method defined in claim 13 wherein said first set of parameters and said second set of parameters each include a pulse duration between approximately 1 msec and approximately 2 seconds.

19. The method defined in claim 13 wherein said dermal tissues contain at least one type of chromophore absorbable by said first pulses of light, said second pulses of light and said third pulses of light, said chromophore being endogenous to said dermal tissues.

20. The method defined in claim 19 wherein said dermal tissues include dermal blood vessels and wherein said chromophore is hemoglobin.

21. The method defined in claim 13 wherein the light of said pulses is incoherent.

22. The method defined in claim 13 wherein said first skin area and said second skin area are disposed proximately to one another, further comprising disposing a template to define said first skin area and said second skin area during the directing of said first pulses of light and said second pulses of light, also comprising disposing said template during the comparing of hair growth along said first skin area with hair growth along said second skin area.

23. A device for applying light energy to a skin surface, comprising:
a housing;
a light source disposed in said housing;
an applicator interface element mounted to said housing for guiding light from said source to the skin surface, said applicator interface element including a hydrogel material.

24. The device defined in claim 23 wherein said applicator interface element includes a block of the hydrogel material.

25. The device defined in claim 23 wherein said applicator interface element includes a polymeric envelope containing a fluid including particles of the hydrogel material.

26. The device defined in claim 23 wherein the light source is in contact with the hydrogel material.
